(19) United States
(12) Plant Patent Application Publication
(10) Pub. No.: US 2014/0283236 P1
(43) Pub. Date: Sep. 18, 2014

(54) Apple Tree Named 'BAB 2000'

(50) ;

(71) Applicant: CASCADE VARIETY MANAGEMENT LLC, Quincy, WA (US)

(72) Inventor: Warren Morgan, Quincy, WA (US)

(73) Assignee: Cascade Variety Management LLC, Quincy, WA (US)

(21) Appl. No.: 13/815,606

(22) Filed: Mar. 12, 2013

(51) Int. Cl. A01H 5/00

(52) U.S. Cl. PLT/161

(57) ABSTRACT

A new strain of apple tree characterized by a brighter fluorescent pink/red color and more complete color coverage than ‘Honeycrisp’. Chronological Maturity development is similar to that of ‘Honeycrisp’, but marketable color is achieved at a much more favorable timing for long-term storage, even when grown under conditions of high nitrogen fertilization.

### LATIN NAME

Malus domestica Borkh.

### VARIETY DENOMINATION

BAB 2000.

### BACKGROUND OF THE VARIETY

The present invention relates to apple trees, specifically to a new and distinct strain or bud mutation of ‘Honeycrisp’ (U.S. Plant Pat. No. 7,197) known as ‘BAB 2000’.

The new apple variety was discovered in the inventor's field near Quincy, Wash. as a whole tree mutation. It was asexually reproduced by grafting through two subsequent generations in orchards owned by the inventor in the same area. The variety ‘BAB 2000’ remained true to type and distinct from the parent variety ‘Honeycrisp’ under identical horticultural conditions. This asexual propagation under controlled conditions clearly proves the maintenance of the characteristics described hereafter which differentiate the new strain from the parent cultivar.

### BRIEF DESCRIPTION OF THE VARIETY

‘BAB 2000’ apple possesses a bright red blush color, covering from 70% to 100% of the fruit, compared to the wide stripe and 30% to 70% red coverage on the parent, and achieves marketable color ten days earlier than the parent even under conditions of very high nitrogen fertilization. Internal maturity development as measured by starch, sugar and pressure is similar to that of ‘Honeycrisp’.

### BRIEF DESCRIPTION OF THE PHOTOGRAPHS

FIG. 1 is a picture of a mature ‘BAB 2000’ apple from a 4th leaf tree and a picture of a ‘Honeycrisp’ apple picked from an adjacent 4th leaf tree, illustrating the superior color of the ‘BAB 2000’ when grown under high nitrogen conditions;

FIG. 2 is a picture of 4th leaf ‘BAB 2000’ trees in the foreground and ‘Honeycrisp’ trees in the background, one week prior to commercial harvest and grown under identical high nitrogen conditions;

FIG. 3 is a picture of ‘BAB 2000’ shoot and leaves;

FIG. 4 is a picture of ‘BAB 2000’ fruits cut in both longitudinal and latitudinal directions; and

FIG. 5 is a picture of a blossom cluster from ‘BAB 2000’ illustrating the floral structure.

The attached photographs are good representations of the plant and its parts but may be affected by the printing process with regard to color.

### DETAILED BOTANICAL DESCRIPTION OF THE VARIETY

The following-detailed botanical description is based on observations made during the 2012 growing season at Quincy, Wash. of 4^{th} leaf trees grown on ‘M9’ rootstock (not patented) and trained to a modified spindle system. All colors are described according to The Royal Horticultural Society Colour Chart. It should be understood that the characteristics described will vary somewhat depending upon cultural practices and climatic conditions, and will vary with location and season. Quantified measurements are expressed as an average of measurements taken from a number of individual plants of the new variety. The measurements of any individual plant or any group of plants of the new variety may vary from the stated average.
Tree:
   Vigor.—Moderate.
   Type.—Upright.
   Habit.—Spreading.
   Height.—3.3 m.
   Trunk diameter (at 20 cm above soil surface).—5 cm.
   Bark texture.—Moderately smooth.
   Bark coloration.—Light grey-brown 201B.
   Lenticels.—3.5 mm long, 0.4 mm wide, greenish white 157D, 3 per cm².
Branch (fruiting branches located at around 1 m above the graft union):
   Diameter.—19 mm.
   Crotch angle.—75° to 90°.
   Lenticels.—1.5 mm long, 0.2 mm wide, greyed-orange 167D, 6 per cm².
One year old shoot:
   Length.—25 cm.
   Color.—Greyed-orange 166B.
Flowers:
   Diameter of fully open flower.—40 mm to 50 mm.
   Relative position of petal margin.—Touching to overlapping.
   Date of full bloom.—April 21 (mid-season).
   Pollination requirement.—Cross-pollination required with similarly timed diploid variety.
Petals:
   Number per flower.13 5.
   Shape.—Obovate.
   Length.—20 mm.
   Apex.—Obtuse.
   Base.—Cuneate.
   Margin.—Smooth to slightly wavy.
   Coloration of upper surface.—Greenish white 155C with red-purple 68D venation.
Stigma:
   Size.—1 mm.
   Color.—Greyed-yellow 160C.
Style:
   Size.—8 mm.
   Color.—Greyed-green 193A.
   Ovary.—Inferior.
Anthers:
   Size.—Length 2 mm, width 1.5 mm.
   Color.—Greyed-yellow 162C.
Peduncle:
   Size.—6 mm.
   Color.—Green 130B.
Leaves:
   Shape.—Ovate.
   Length.—6 cm to 9 cm.
   Width.—4 cm to 6.5 cm.
   Blade margin.—Doubly serrate.
   Apex.—Acuminate.
   Base shape.—Rounded.
   Color of upper surface.—Green 137A.
   Color of lower surface.—Greyed-green 191C.
   Venation.—Macrophyllous, green 137A.
Petiole:
   Length.—2.5 cm to 3.2 cm.
   Diameter.—3 mm.
   Coloration.—Red-purple 71A blending to green 132A.
Fruit:
   Maturity when described.—Ripe.
   Diameter.—9 cm.
   Weight.—340 g.
   General shape in profile.—Oblate.
   Position of maximum diameter.—Toward stem end.
   Crowning at calyx end.—Minimal.
   Size of eye.—6 mm.
   Aperture of eye.—Closed.
   Length of sepal.—5 mm.
   Bloom of skin.—Moderate.
   Greasiness of skin.—Light.
   Color of skin.—Red 52A.
   Amount of russet around stalk cavity.—Slight.
   Amount of russet on cheeks.—None.
   Area of russet around eye basin.—None.
   Length of stalk.—19 mm.
   Thickness of stalk.—3 mm.
   Depth of stalk cavity.—17 mm.
   Width of stalk cavity.—3.8 cm at shoulders.
   Depth of eye basin.—12 mm.
   Width of eye basin.—2.5 cm.
   Firmness of flesh.—12.8 lb.
   Flesh texture.—Crisp.
   Aroma.—Sweet spicy, similar to ‘Honeycrisp’.
   Juiciness.—Juicy.
   Brix.—11.6.
   Flesh coloration.—White 155B.
   Stem coloration.—Grey-brown 199A.
Harvest:
   Time for harvest.—Mid-September.
   Amount of fruit produced per tree per harvest.—22 kg.
   Disease resistance/susceptibility.—Very susceptible to mildew (similar to ‘Honeycrisp’).
   Market use.—Fresh.

I claim:

1. A new and distinct apple tree substantially as described and illustrated herein, characterized by improved fruit color and brightness, which is maintained under conditions of high nitrogen fertilization.
